(19) United States
(12) Patent Application Publication
(10) Pub. No.: US 2022/0088572 A1
(43) Pub. Date: Mar. 24, 2022

(54) Nano-Structured Composite Materials for Chemical Air Pollutant and Odor Removal from Air

(71) Applicants: Xiwang Qi, Scottsdale, AZ (US); Mei Jiang, Scottsdale, AZ (US)

(72) Inventors: Xiwang Qi, Scottsdale, AZ (US); Mei Jiang, Scottsdale, AZ (US)

(73) Assignee: Freshean Corp., SCOTTSDALE, AZ (US)

(21) Appl. No.: 16/948,463

(22) Filed: Sep. 18, 2020

(51) Int. Cl. B01J 21/18; B01D 53/86; A61L 9/014; B01J 29/70; B01J 29/40; B01J 29/08; B01J 29/18; B01J 23/10

(52) U.S. Cl. CPC B01J 21/18; B01D 2257/2064; A61L 9/014; B01J 29/7007; B01J 29/40; B01D 53/8662; B01D 53/8628; B01D 53/8675; B01D 53/865; B01D 53/8615; B01J 29/082; B01J 29/18; B01D 53/8678; B01J 29/7003; B01D 53/8634; B01J 23/10; B01D 53/8668; B01D 2255/92; B01D 2255/702; B01D 2255/206; B01D 2255/502; B01D 2258/06; B01D 2257/708; B01D 2257/306; B01D 2257/2025; B01D 53/8637

(57) ABSTRACT

Described is related to nano-structured composite materials for removing harmful chemical air pollutants and odors from the air to prevent people from breathing in disease-causing chemicals and provide them with clean indoor air. The nano-structured composite materials comprise nano-catalysts embedded in the pores of nano-structured substrate materials selected from the group consisting of nano-porous carbon, nano-porous rare earth oxide, nano-porous zeolite, nano-porous alumina and nano-porous silica. The nano-scale synergy of nano-catalysts and nano-structured substrate materials provides effective air filtration materials for the complete trapping and elimination of the full spectrum of chemical air pollutants including both organic and inorganic compounds and odors for indoor spaces, which HEPA or activated carbon filters cannot achieve.

### CROSS-REFERENCE TO RELATED APPLICATION

This application claims priority of Provisional Application Ser. No. 62/909,238 filed in the United States Patent and Trademark Office on Oct. 2, 2019, which addresses the same subject matter.

### FIELD OF THE INVENTION

This invention is related to nano-structured composite materials, consisting of nano-catalysts supported on nano-structured substrate materials, for removing harmful chemical pollutants and odors from air at room temperature and providing protection for people from breathing in disease-causing chemicals.

### BACKGROUND OF THE INVENTION

Air pollution has been one of the biggest threats to human health in recent decades. According to the US EPA, indoor air pollution is 2-5 times higher than outdoors. The World Health Organization reported that 3.8 million deaths per year are attributed to household air pollution worldwide. Air filters have been one of the major measures in recent years used to battle against air pollution. These include HVAC filters in homes and commercial buildings, cabin air filters in vehicles and airplanes, HEPA filters in air purifiers, as well as facemasks. However, all those filters, made from synthetic fibers, can only trap particles such as pollen and dust based on a “sieving effect”, and unfortunately cannot remove the more dangerous gaseous chemical pollutants which are present in the air as single molecules that are 1000 times smaller than the holes of even the best HEPA filter so they pass right through.

Chemical air pollutants are very common inside vehicles, airplanes, homes, and commercial buildings and originate from engine exhaust, wild fire smoke, heating and cooking smoke, cigarette smoke, as well as emissions from home interior materials, plastic wallpapers, synthetic foams, adhesives, paints, furniture, carpet, air fresheners, hairspray, cleaning supplies, pesticides, etc. These chemical pollutants are known to be very toxic and include many carcinogens like formaldehyde, benzene, butadiene and polycyclic aromatic hydrocarbons, as well as other disease causing chemicals such as carbon monoxide, nitric oxide, nitrogen dioxide, sulfur dioxide, hydrogen sulfide, propene, butane, acetone, toluene, xylene, ammonia, alcohols, chlorine, mercaptans, and many other volatile organic compounds. Exposure to those chemicals can cause common ailments such as headaches, coughs, nausea, dizziness, fatigue, depression, while prolonged inhalation can cause permanent damage to the lungs, liver, heart, brain and other body systems through diseases such as leukemia and other forms of cancer. Children, pregnant women, and the elderly are especially vulnerable because of their weaker immune systems.

For example, a common practice for many people is the use of air fresheners like Febreze®, Glade® and Air Wick® to cover up bad odors or even chemical smells. What many people may not realize is that these products are all chemically-based fragrances, so not only do such practices fail to actually remove odors or harmful substances from the air, but they also add new chemicals into the air which presents a threat to human health.

In recent years, activated carbon has been used as a major adsorbent in air filters to remove certain organic air pollutants. For example, U.S. Pat. No. 6,773,477 describes a portable air purifier for passenger cars which includes packets of desiccant, carbon particles and a HEPA filter. U.S. Pat. No. 6,680,028 describes an air purifier filter with activated charcoal, potassium permanganate, potassium hydroxide, etc. US Patent Application 20130040804 discloses a passenger vehicle air filter containing granular activated carbon and Fe exchanged zeolite. However, the efficacy and capacity of activated carbon is limited in dealing with the wide range of chemical pollutants encountered in vehicles, homes and buildings, which include both organic and inorganic chemical pollutants. The growth of both organic and inorganic chemical pollutants in proximity to humans has been an ever-increasing trend in the past few decades. Activated carbon is especially ineffective at removing some of the major gaseous air pollutants in vehicles or homes such as carbon monoxide, nitric oxide, nitrogen dioxide, sulfur dioxide, hydrogen sulfide, formaldehyde, ammonia, etc. Therefore, there is an urgent need for an air filtration material that can efficiently and effectively remove the full spectrum of chemical air pollutants including both organic and inorganic chemical pollutants to provide maximum protection of people's health.

### BRIEF SUMMARY OF THE INVENTION

This invention is related to nano-structured composite materials, consisting of nano-catalysts supported on nano-porous substrate materials in the form of a “Nano-in-Nano”configuration, for the removal of harmful chemical pollutants and odors from the air at room temperature. The nano-scale integration of nano-catalysts with nano-structured substrate materials as well as the tailored molecular-level material matching achieved between nano-catalysts and substrate materials create extremely active trapping and catalytic conversion sites for the elimination of the full spectrum of harmful chemical pollutants including both organic and inorganic compounds and odors from air via physical and chemical synergy, for which HEPA filters and/or activated carbon filters cannot achieve otherwise. The nano-structured composite materials of this invention can be utilized in various applications for different air purification needs, such as portable air purifiers, residential air purifiers, commercial air purifiers, HVAC air filters, vehicle and airplane cabin air filters, as well as personal facemasks.

### DETAILED DESCRIPTION OF THE INVENTION

This invention is related to nano-structured composite materials for removing harmful chemical air pollutants and odors from air, consisting of nano-catalysts supported on nano-porous substrate materials. The nano-porous substrate materials include at least one of nano-porous carbon, nano-porous zeolite, nano-porous rare earth oxide, nano-porous silica and nano-porous alumina, with nano-sized catalysts distributed and anchored on the surface of internal nano-pores of those nano-porous substrate materials.

The above nano-structured composite materials are made by mixing nano-catalysts with at least one substrate materials selected from raw carbon materials, rare earth oxide, silica, alumina and zeolite, as well as ceramic fillers, pore formers, organic binder and solvents together into a slurry or paste, followed by spraying or extrusion or tape-casting or molding to form various shapes, or by coating on existing substrate of various forms, then followed by drying, calcination and activation at high temperatures to create highly porous and active filter materials. The final physical formats of nano-structured composite materials made by this approach include fine powders, small grains, pellets, laminated sheets in cylindrical configuration or corrugated/pleated configuration, honeycomb monoliths, molded pieces in the form of ball, cylinder, cube, and hexagonal prism with interconnected internal porous structure.

The above nano-structured composite materials can also be made by mixing liquid catalyst precursors with at least one substrate materials selected from liquid carbon precursor, rare earth oxide, silica, alumina and zeolite, as well as ceramic fillers, pore formers, organic binder and solvents together into a slurry or paste, followed by spraying or extrusion or tape-casting or molding to form various shapes, or by coating on existing substrate of various forms, then followed by drying, calcination and activation at high temperatures to create highly porous and active filter materials. The final physical formats of nano-structured composite materials made by this approach include fine powders, small grains, pellets, laminated sheets in cylindrical configuration or corrugated/pleated configuration, honeycomb monoliths, molded pieces in the form of ball, cylinder, cube, and hexagonal prism with interconnected internal porous structure.

The raw carbon materials are selected from the group consisting of coal charcoal, bamboo charcoal, coconut shell charcoal, willow bark charcoal and wood dust charcoal. The liquid carbon precursor is selected from epoxy resin, phenolic resin and Furan resin. The rare earth oxide is selected from the group consisting of yttrium oxide, lanthanum oxide, cerium oxide, praseodymium oxide, samarium oxide, and rare earth doped oxides, including yttria-zirconia and ceria-zirconia, and combinations. The nano catalysts are selected from the group consisting of iron, cobalt, nickel, copper, zinc, manganese, magnesium, potassium, sodium, calcium, barium, titanium, platinum, palladium, rhodium, ruthenium, silver, cerium, and their corresponding oxides and combinations. The liquid catalyst precursors are selected from the corresponding salt solution group consisting of iron, cobalt, nickel, copper, zinc, manganese, magnesium, potassium, sodium, calcium, titanium, platinum, palladium, rhodium, ruthenium, silver, cerium, and combinations. The ceramic fillers are selected from silica, alumina, rare earth doped silica, rare earth doped alumina, aluminosilicate, silicon carbide, cordierite, mullite, or combinations. The zeolite is selected from the group consisting of 3A zeolite, 4A zeolite, 5A zeolite, 13X zeolite, Beta zeolite, Pentasil zeolite and Mordenite zeolite. The pore formers are selected from cellulose powder, polyethylene powder, polypropylene powder, corn starch and potato starch. The organic binder is selected from methyl cellulose and polyethylene glycol.

The chemical air pollutants that can be removed by the nano-structured composite materials of this invention include, but are not limited to, carbon monoxide, nitric oxide, nitrogen dioxide, sulfur dioxide, hydrogen sulfide, ammonia, chlorine, ozone, formaldehyde, benzene, acetaldehyde, butadiene, propene, acetone, toluene, xylene, mercaptans and polycyclic aromatic hydrocarbons. The odors that can be removed by the nano-structured composite materials of this invention include, but are not limited to, cigarette smoke, wildfire smoke, alcoholic beverages, body odors, perfumes, bathroom and toilet smells, dirty laundry smells, sewer odors, mold smells, garlic and onion odors, rotten and burned food, diesel and gasoline fumes, paint odors, moth balls, pet, fish and poultry odors, etc.

In one embodiment, raw carbon material powder, rare earth oxide powder, zeolite powder, nano-sized catalysts, ceramic filler powders, pore former and organic binder are mixed together with water into a paste and is extruded into pellets with size from 1 mm to 5 mm. The pellets are then dried at 50° C. to 160° C., calcined at 400° C. to 900° C., and activated at 500° C. to 900° C. by agents chosen from water vapor, carbon dioxide, sodium hydroxide, potassium hydroxide, magnesium hydroxide, calcium hydroxide, sodium carbonate, potassium carbonate, magnesium carbonate and calcium carbonate.

In another embodiment, the liquid carbon precursor, the liquid catalyst precursors, rare earth oxide powder, zeolite powder, ceramic filler powders, pore former and organic binder are mixed together with water into a paste and is extruded into pellets with size from 1 mm to 5 mm. The pellets are then dried at 50° C. to 160° C., calcined at 400° C. to 900° C., and activated at 500° C. to 900° C. by agents chosen from water vapor, carbon dioxide, sodium hydroxide, potassium hydroxide, magnesium hydroxide, calcium hydroxide, sodium carbonate, potassium carbonate, magnesium carbonate and calcium carbonate.

In another embodiment, the raw carbon material powder, alumina powder, the liquid catalyst precursors, ceramic filler powders, pore former and organic binder are mixed together with isopropanol into a slurry and is spray dried into powders or small grains with size ranging from 18 mesh to 200 mesh. The powders or grains are then calcined at 400° C. to 900° C., and activated at 500° C. to 900° C. by agents chosen from water vapor, carbon dioxide, sodium hydroxide, potassium hydroxide, magnesium hydroxide, calcium hydroxide, sodium carbonate, potassium carbonate, magnesium carbonate and calcium carbonate. The resulting powders or grains are further coated onto a synthetic non-woven fiber sheet, which is then laminated with another non-coated fiber sheet to form a sandwiched structure with nano-structured composite powders held in-between. The laminated sheet can be used as it is in flat format or can be stacked onto each other with corrugated sheet in between to form air channels, or can be made into pleated form, as chemical and odor air filter.

1. Nano-structured composite materials for removing chemical air pollutants and odors from air at room temperature comprise nano-catalysts supported on nano-structured substrate materials in the form of “Nano-in-Nano” configuration wherein nano-sized catalysts distributed and anchored inside the nano-pores of the substrate materials which include at least one of nano-porous carbon, nano-porous rare earth oxide, nano-porous zeolite, nano-porous alumina, nano-porous silica, or combinations thereof.

2. The nano-structured composite materials according to claim 1, wherein said chemical air pollutants include carbon monoxide, nitric oxide, nitrogen dioxide, sulfur dioxide, hydrogen sulfide, ammonia, chlorine, ozone, formaldehyde, acetaldehyde, butadiene, propene, butane, acetone, benzene, phenol, toluene, xylene, methylene chloride, tetrachloroethylene, mercaptans and polycyclic aromatic hydrocarbons.

3. The nano-structured composite materials according to claim 1, wherein said odors include cigarette smoke, wildfire smoke, alcoholic beverages, body odors, perfumes, bathroom and toilet smells, dirty laundry smells, mold smells, locker room odors, sewer odors, garlic and onion odors, rotten and burned food, diesel and gasoline fumes, paint odors, moth balls, pet, fish and poultry odors.

4. The nano-structured composite materials according to claim 1, wherein said nano-catalysts are selected from a group consisting of iron, cobalt, nickel, copper, zinc, manganese, magnesium, sodium, potassium, calcium, barium, titanium, platinum, palladium, rhodium, ruthenium, silver, cerium, their corresponding oxides, and combinations thereof.

5. The nano-structured composite materials according to claim 1, wherein said nano-porous carbon is derived from raw carbon materials selected from a group consisting of coal charcoal, bamboo charcoal, coconut shell charcoal, willow bark charcoal, wood dust charcoal and liquid carbon precursors.

6. The nano-structured composite materials according to claim 5, wherein said liquid carbon precursors are selected from epoxy resin, phenolic resin and Furan resin.

7. The nano-structured composite materials according to claim 1, wherein said rare earth oxide is selected from a group consisting of yttrium oxide, lanthanum oxide, cerium oxide, praseodymium oxide, samarium oxide, and rare earth doped oxides including yttria-zirconia and ceria-zirconia, and combinations thereof.

8. The nano-structured composite materials according to claim 1, wherein said zeolite is selected from a group consisting of 3A zeolite, 4A zeolite, 5A zeolite, 13X zeolite, Beta zeolite, Pentasil zeolite and Mordenite zeolite, and combinations thereof.
